(19) United States
(12) Patent Application Publication
(10) Pub. No.: US 2009/0082713 A1
(43) Pub. Date: Mar. 26, 2009

(54) METHOD OF ENHANCING IONTOPHORETIC DELIVERY OF A PEPTIDE

(76) Inventor: Phillip M. Friden, Bedford, MA (US)

Correspondence Address: ELMORE PATENT LAW GROUP, PC, 515 Groton Road, Unit 1R, Westford, MA, 01886, US

(21) Appl. No.: 12/234,071

(22) Filed: Sep. 19, 2008

Related U.S. Application Data
(60) Provisional application No. 60/973,956, filed on Sep. 20, 2007.

(51) Int. Cl. A61N 1/30; A61K 38/16; A61P 43/00

(52) U.S. Cl. 604/21; 514/12

(57) ABSTRACT

The present invention provides methods for the administration of a peptide to a body surface of the patient comprising treating said body surface by microporation and iontophoretically administering the peptide to the body surface. The present invention also encompasses a method of transdermally administering a peptide to the skin of a patient comprising treating the skin with microporation and iontophoretically administering said peptide to the skin. In one embodiment, the body surface or skin is microporated using one or more microneedles.

### RELATED APPLICATION

This application claims the benefit of Provisional Application No. 60/973,956 filed on Sep. 20, 2007. The entire teachings of the above application are incorporated herein by reference.

### BACKGROUND OF THE INVENTION

An iontophoretic delivery system is an example of a drug delivery system that releases drug at a controlled rate to the target tissue upon application. The advantages of systems wherein drug is delivered locally via iontophoresis are the ease of use, being relatively safe, and affording the interruption of the medication by simply stopping the current and/or peeling off or removing it from the skin or other body surface whenever an overdosing is suspected. The total skin surface area of an adult is about 2 m². In recent years iontophoretic delivery of drugs has attracted wide attention as a better way of administering drugs for local as well as systemic effects. The design of iontophoretic delivery systems can usually be such that the side effects generally seen with the systemic administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions into a target tissue, such as through the stratum corneum and into the epidermal/dermal layers of the skin. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's body to form a closed circuit between one of the electrodes (referred to herein alternatively as a “working” or “application” or “applicator” electrode) which is positioned at the site of drug delivery and a passive or “grounding” electrode affixed to a second site on the body surface to enhance the rate of penetration of the medicament into the tissue adjacent to the applicator electrode.

U.S. Pat. No. 6,477,410 issued to Henley et al. describes the use of iontophoresis for drug delivery. It would be advantageous to improve the permeation of high molecular weight drugs such as proteins by iontophoretic delivery.

### SUMMARY OF THE INVENTION

It has now surprisingly been found that microporation combined with iontophoretic administration of a protein resulted in improved transdermal delivery of the protein. As shown in Example 1 below, in the hairless rat model, the combination of microneedle treatment with iontophoretic administration of salmon calcitonin increased the amount of protein that permeated the skin by about four times compared to the use of iontophoresis alone.

The present invention provides methods for the administration of a peptide to a body surface of the patient comprising microporating the body surface and iontophoretically administering the peptide to said body surface.

The invention is also directed to methods of administering a peptide to the body surface of a patient in need thereof comprising microporating the body surface with one or more microneedles and iontophoretically administering the peptide to said body surface.

In another embodiment, the present invention is directed to a method of transdermally administering a peptide to the skin of the patient comprising microporating the body surface with one or more microneedles and iontophoretically administering the peptide into the skin of the patient. In one embodiment, the skin is pretreated with microporation using a microneedle followed by administration of the drug using iontophoresis.

The present invention also encompasses a method of transdermally administering a peptide to the skin of a patient comprising microporating the skin of said patient with one or more microneedles while concurrently iontophoretically administering said peptide into the skin.

### BRIEF DESCRIPTION OF THE DRAWINGS

FIG. 1A is a drawing of a titanium microneedle array bent out of plane.

FIG. 1B shows the dimensions (μm) of a titanium microneedle array and of each microneedle.

FIG. 1C is a plot of the plasma concentration (ng/ml) over time (min) of salmon calcitonin delivered using microneedles alone, iontophoresis alone or the combination of microneedles and iontophoresis in the hairless rat model.

### DETAILED DESCRIPTION OF THE INVENTION

As used herein, the word “a” or “an” is meant to encompass one or more unless otherwise specified. For example, “a microneedle” is intended to encompass one or more microneedles.

The invention is directed to methods of administering a peptide to a body surface comprising microporating the body surface and iontophoretically administering said peptide to the body surface. In one embodiment, the body surface is microporated using one or microneedles. In another embodiment, the body surface is the skin. In one embodiment, the body surface is microporated prior to iontophoretic administration of the peptide. In yet other embodiment, the body surface is microporated using one or more hollow or porous microneedles while concurrently iontophoretically administering the peptide.

As used herein, the term “peptide” is meant to encompass proteins, peptide drugs as well as amino acid drugs (such as the beta lactam antibiotics including the penicillins and the cephalosporins). Peptides have a molecular weight of at least about 500 Daltons (Da). The term “peptide” is also meant to include proteins or peptide drugs which have been chemically modified. Such chemical modifications include, for example, replacement of an amino acid with a different amino acid or other group and/or addition of a functional group and/or a chemical modifier. In one embodiment, the peptide administered according to a method of the invention has a molecular weight of at least about 500 Da. In another embodiment, the peptide administered according to the inventive method has a molecular weight of at least about 1000 Da. In a further embodiment, the peptide administered according to a method of the invention has a molecular weight of at least about 3000 Da. In another embodiment, the molecular weight of the peptide is at least about 10,000 Da. In yet another embodiment, the molecular weight of the peptide is at least about 100,000 Da.

In one embodiment, the peptide administered according to a method of the invention is a therapeutic protein. Therapeutic proteins, include but are not limited to, cytokines, hormones and antibodies. In another embodiment, the peptide administered according to a method of the invention is selected from the group consisting of a fusion protein and an antibody.

Proteins and peptide drugs that may be used in the method of the present invention include, but are not limited to, Luteinizing hormone-releasing hormone (LHRH), Somatostatin, Bradykinin, Goserelin, Somatotropin, Buserelin, Platelet-derived growth factor, Triptorelin, Gonadorelin, Asparaginase, Nafarelin, Bleomycin sulfate, Leuprolide Chymopapain, Growth hormone-releasing factor, Cholecystokinin, Chorionic gonadotropin, Insulin, Corticotropin (ACTH), Calcitonin (e.g., eel, salmon, Erythropoietin human), Glucagon, Calcitonin gene related peptide, Hyaluronidase Interferons (e.g., alpha, beta and gamma), Endorphin (alpha, beta, and and gamma), Interleukins (e.g., IL-1, IL-4, IL-6, IL-2 and IL-10), Thyrotropin-releasing hormone, CSIF (cytokine synthesis inhibitory factor), NT-36 (N-[[(s)-4-oxo-2-azetidinyl]carbonyl]-L-histidyl-L-prolinamide, Liprecin, Menotropins, Pituitary hormones (e.g., HGH, HMG, HCG, desmopressin acetate, etc.), Urofollitropin (Follicle Stimulating Hormone), desmo-pressin acetate, etc., Leutinizing hormone (LH), aANF growth factor releasing factor, leutinizing hormone (LH), LH releasing hormone, Melanocyte-stimulating hormone (alpha, beta and gamma), Vasopressin, Streptokinase, ACTH analogs, Tissue plasminogen activator, Atrial natriuretic peptide, ANP clearance inhibitors, Urokinase, Angiotensin II antagonists, Bradykinin potentiator B, Bradykinin antagonists, Bradykinin potentiator C, CD4, Ceredase, Brain-derived neutrotrophic factor, Colony stimulating factors, Cystic fibrosis transmembrane conduce regulator (CFTR), Enkephalins, Fab fragments, IgE peptide suppressors, Chorionic gonadotoropin, Insulin-like growth factors, Ciliary neutrotrophic factor, Neurorophic factors, Parathyroid hormone, Corticotropin releasing factor, Prostaglandin antagonists, Granulocyte colony stimulating factor, Pentigetide, Protein C, Protein S, Thymosin a-1, Thrombolytics, Tumor necrosis factor alpha (TNF-a), Multilineage colony stimulating factor, Macrophage-specific colony stimulating factor, Vaccines, Vasopressin antagonist, Colony stimulating factor 4, a-1 Anti-trypsin, Adenosine deaminase, Epidermal growth factor, Amylin, Atrial natriuretic peptide, Enkephalin leu, B-Glucocerebrosidase, Enkephalin met, Bone morphogenesis protein 2, Factor IX, Bombesin, Factor VIII, Bactericidal/Permeability increasing protein, Follicular gonadotropin releasing peptide, Hirudin, G-1128, IEV inhibitor peptide, Gastrin-releasing peptide, Inhibin-like peptide, Glucagon, Insulin, Insulinotropin, Growth hormone releasing factor, Lipotropin, Macrophage-derived neutrophil chemotaxis factor, Heparin binding neurotrophic factor, Melatonin, Tryptophan hydroxylase, Fibroblast growth factor, Midkine, Neurophysin, Somatostatin, Neurotrophin-3, Nerve growth factor, Oxytocin, Phospholipase A2, Soluble IL-1 receptor, Thymidine kinase, Thymosin alpha one, soluble TNF receptor, Tissue plasminogen activator, Transforming growth factor beta, TSH-releasing hormone, Thyroid stimulating hormone (TSH), Vasopresssin and Vasotocin. Proteins that may be used according to the present invention include antibodies. In the present invention, antibodies include, but are not limited to, polyclonal, monoclonal, chimeric, single-chain, humanized and human antibodies, as well as various fragments thereof such as Fab fragments and fragments produced from specialized expression systems.

In one embodiment, a current density sufficient for permeation into a body surface is applied. In another embodiment, a current density sufficient for permeation through the stratum corneum is applied. In one embodiment, a current density of about 0.001 mA/cm² to about 2.0 mA/cm² is applied. In yet another embodiment, a current density of about 0.01 mA/cm² to about 1 mA/cm² is applied. In a further embodiment, a current density of about 0.05 mA/cm² to about 0.5 mA/cm² is applied. In an additional embodiment, a current density from about 0.1 mA/cm² to about 0.5 mA/cm² is applied.

The iontophoresis can be applied for a sufficient time to achieve an effective amount of permeation. For example, a sufficient time for application is a time from about 1 minute to about 4 hours. In one embodiment, iontophoresis is applied for a time from about 5 minutes to about 2 hours. In yet another embodiment, iontophoresis is applied for a time from about 10 minutes to about 90 minutes. In a further embodiment, iontophoresis is applied from about 10 minutes to about 1 hour.

In one embodiment, the peptide is formulated with a pharmaceutically acceptable carrier or excipient. As used herein, the term “pharmaceutically acceptable carrier or excipient” means any non-toxic diluent or other formulation auxiliary that is suitable for use in iontophoresis. Examples of pharmaceutically acceptable carriers or excipients include, but are not limited to, solvents, cosolvents, solubilizing agents (such as sorbitol and glycerin), buffers, pharmaceutically acceptable bases, alcohols such as benzyl alcohol and viscosity modulating agents such as cellulose and its derivatives. The formulation may further comprise a chemical permeation enhancer. A “permeation enhancer” is a material which achieves permeation enhancement or an increase in the permeability of the body surface to a pharmacologically active agent. Examples of such permeation enhancers include, but are not limited to, N-acetylcysteine, urea, salicylic acid, linoleic acid, benzoic acid, cyclodextrin, dimethyl sulfoxide, dimyristoyl phosphatidylserine, and the like. In another embodiment, the formulation may contain stabilizers such as antioxidants (EDTA, sodium sulfites, ascorbic acid, vitamin E, BHT, etc.) and/or an alcohol. In another embodiment, the formulation comprising the protein may contain a preservative such as benzalkonium chloride, parabens, etc. In a further embodiment, the formulation may contain an agent that affects protein binding including, but not limited to, linolenic acid, dimyristoyl phosphatidyl glycerol (DPMG), a polysorbate and dimyristoyl phosphatidyl choline (DPMC). The peptide can be administered in a therapeutically effective amount. A “therapeutically effective amount” is an amount of peptide that is sufficient to prevent development of or alleviate to some extent one or more of a patient's symptoms of a disease being treated or to elicit a desired biological or medical response in a subject.

In one embodiment, the peptide is iontopheretically administered using an iontophoretic delivery device. Examples of iontophoretic delivery devices useful with the compositions and methods of the invention include, but are not limited to, those described in U.S. Pat. Nos. 6,148,231, 6,385,487, 6,477,410, 6,553,253, 6,792,306, 6,895,271, 7,016,724 and 7,127,285, all incorporated herein by reference. An example of an applicator which can be used with a formulation of the invention comprises an active electrode adhered to an open cell polymer foam or hydrogel. Another applicator which has been developed for use with a device for iontophoretic delivery of an agent to a treatment site comprises an applicator head having opposite faces and including an active electrode and a porous pad (such as a woven or non-woven polymer, for example, a polypropylene pad); a margin of the applicator head about the active electrode having a plurality of spaced projections there along; the porous pad and the applicator head being ultrasonically welded to one another about the margin of the head with the electrode underlying the porous pad; and a medicament or a medicament and an electrically conductive carrier therefor carried by the porous pad in electrical contact with the electrode. In one embodiment, the formulation is iontophoretically administered using carbon electrodes, silver-silver chloride electrodes or silver coated carbon electrodes.

In one embodiment, the body surface is selected from the group consisting of the skin, the nail plate, the eyes, the ears and a mucous membrane.

Microporation refers to the formation of micropores on a body surface. A micropore in the skin means a small breach or pore formed in the stratum corneum within a selected area of the skin to decrease the barrier properties of the stratum corneum. Microporation may be achieved using any suitable method including, but not limited to, the use of a microneedle, thermal poration, radiofrequency ablation, laser ablation, and sonophoresis (with or without the use of dyes or other energy absorbing materials to assist in the ablation and removal of the stratum corneum).

In one embodiment, microporation of the body surface is achieved using one or more microneedles. The length and density of the microneedle as well as the thickness or diameter of the needles can vary depending on the location of the targeted treatment site underlying the skin surface. In one embodiment, the microneedle has a height of about 2 millimeters (mm) or less and/or are about 50 to about 300 μm in diameter when such structures are cylindrical in nature. In an additional embodiment, the microneedle has a diameter of about 100 to about 200 μm. Non-cylindrical structures are also encompassed by the term microneedle; such microneedles are of comparable cross-sectional length or cross-sectional area and include pyramidal, rectangular, octagonal, wedged, and other geometrical shapes. Microneedles have been described, for example, in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated by reference herein. In another embodiment, the microneedle may protrude from a substrate by the height of 2 mm or less. In another embodiment, the microneedle has a height of about 1 mm or less. In yet another embodiment, the microneedle has a height from about 100 to about 1 mm. In yet an additional embodiment, the microneedle has a height from 150 to 900 μm. In another embodiment, the microneedle has a height of about 300 to 800 μm. In one embodiment, the microneedle is of sufficient height to penetrate beyond the stratum corneum to an underlying layer of skin. In another embodiment, the microneedle is of sufficient height to pass into the dermis but not a height great enough to stimulate nerves in deeper tissue and/or cause pain when applied or inserted into the body surface. In another embodiment, the ratio length to width (at the base of the microneedle) is from about 0.5 to about 16.0.

The number of microneedles that can be used in the inventive method is one or more. In one embodiment, the method employs more than one microneedle. In another embodiment, the method employs more than five microneedles. In a further embodiment, the method employs more than ten microneedles. In yet another embodiment, the method employs more than about one hundred microneedles. In other embodiments, a microneedle array is used. A microneedle array has more than two microneedles and can include tens, hundreds, or thousands of needles. The density of microneedles in the microneedle array may be from about 1 to about 1000 needles per cm². The microneedles can be attached and/or arranged in a pattern or randomly over the surface of a substrate. As used herein the “substrate” of a microneedle device includes the base to which the microneedles are attached or integrally formed. Such substrates can be constructed from a variety of materials, including, for example, metals, ceramics, semiconductors, organics, polymers, and composites. In one embodiment, the substrate and/or microneedles, as well as other components, are formed from flexible materials to allow the device to fit the contours of the body surface. Microneedles include solid microneedles, hollow microneedles and porous microneedles.

A microneedle can be made of any suitable material allowing it to penetrate the body surface. Suitability of the material can be determined by considering the compatibility of the material with the body surface or any agent that is in contact with the microneedle, such as the drug or protein to be administered or the formulation comprising the drug as well as the mechanical properties of the material as they pertain creating mechanically robust structures. The microneedles can be formed of a non-conductive material (e.g., a plastic material or a metal material coated with a non-conductive material). The microneedles can also be formed of conductive materials and coated with a non-conductive layer. Suitable materials include, for example, glassy materials, metals, ceramics, semiconductors, organics (such as sugars), polymers including biodegradable polymers and plastics, composites, and combinations of such materials. Sugars include, for example, maltose (Miyano et al. (2005), Biomedical Microdevices, 7(3): 185-8). Metals include pharmaceutical grade stainless steel, gold, titanium, nickel, iron, gold, tin, chromium, copper, alloys of these or other metals, silicon, silicon dioxide, and polymers. Biodegradable polymers include polymers of hydroxy acids such as lactic acid and glycolic acid polylactide, polyglycolide, polylactide-co-glycolide, and copolymers with PEG, polyanhydrides, poly(ortho)esters, polyurethanes, poly(butyric acid), poly(valeric acid), poly(lactide-co-caprolactone) and the like. Non-biodegradable polymers include polycarbonate, polymethacrylic acid, ethylenevinyl acetate, polytetrafluorethylene (TEFLON), polyesters and the like. Suitable polymeric materials include acrylonitrile-butadiene-styrenes, polyphenyl sulfides, polycarbonates, polypropylenes, acetals, acrylics, polyetherimides, polybutylene terephthalates, polyethylene terephthalates and the like.

One aspect of the invention is directed to a method of transdermally administering a peptide to the skin of a patient comprising microporating the skin with one or more microneedles and iontophoretically administering the peptide. Microneedles that may be used in a method of the invention include solid microneedles as well as microneedles possessing one or more orifices through which drug can be delivered into the skin. Microneedles with one or more orifices include hollow and porous microneedles. A hollow microneedle can have one or more substantially annular bores or channels through the interior of the microneedle structure, having a diameter sufficiently large to permit passage of fluid and/or solid materials through the microneedle. The annular bores may extend throughout all or a portion of the needle in the direction of the tip to the base, extending parallel to the direction of the needle or branching or exiting at a side of the needle, as appropriate. The diameter of the bore of the hollow microneedle can be about 5 μm to about 100 μm. Porous microneedles have pores or voids throughout at least a portion of the microneedle which are sufficiently large and sufficiently interconnected to permit passage of fluid and/or solid materials through the microneedle. The diameter of the pore of the porous microneedle can be about 5 μm to about 20 μm.

Another embodiment of the invention is directed to a method of transdermally administering a peptide to the skin of a patient comprising microporating the skin with a microneedle while concurrently administering the peptide into the skin using iontophoresis. According to this aspect of the invention, microporation and peptide administration occur concurrently. Hollow or porous microneedles can be used to create micropores in the skin while at the same time administering a peptide into the skin (and through the microneedle).

Solid microneedles may also be used to concurrently microporate the skin and iontophoretically administer the peptide if the solid microneedles are fabricated of a material that dissolves upon contact with fluid within and contains the peptide. An example of a material that dissolves upon contacting the skin and can contain a peptide is a bioresorbable polymer such as polylactic acid. Solid microneedles can also be used according to this aspect of the invention when they have one or more indentations along their surface which create a channel or trough on the needle surface along which fluid could flow. For example, a solid microneedle can have a “C” shaped indentation that runs along the length of the needle through which fluid flows. The diameter of the indentation of the solid microneedle can be about 5 μm to about 100 μm.

In another embodiment, concurrent drug delivery and microporation are achieved with a microneedle in contact with the skin, a drug reservoir in contact with the microneedles and an electrode in contact with the drug reservoir, wherein the drug reservoir comprises a peptide. In a further embodiment, concurrent drug delivery and microporation are achieved.

In one embodiment, an iontophoretic patch is utilized. The patch may include a rigid boundary surrounding an array of microneedles enabling, upon application, the skin surrounded by the boundary to present itself. In another embodiment, a microneedle is attached to a slightly concave-shaped elastomeric backing attached to the iontophoretic patch and acts as a suction cup. Upon actuation by the user, the target skin area is pulled into the concavity and against the microneedles attached to the more rigid backing material.

In a further embodiment, the substrate upon which the needles are attached may be combined with a delivery device. For example, the finger mounted devices disclosed in U.S. Pat. Nos. 6,792,306 and 6,735,470 may be provided with substrates containing needles of selected sizes and configurations to penetrate through the high electrically resistant layers of the skin to supply medicament to the targeted treatment site. Alternatively, the device disclosed in U.S. Pat. No. RE37796, may also use substrates comprising microneedles described herein. In all instances, by forming a multiplicity of low electrically resistant micropores through the higher electrically resistant layer or layers of the skin, the peptide can be driven from the supply matrix or drug reservoir through the microneedles directly to the targeted treatment site bypassing the high electrically resistant layers of skin.

Additional devices that can be used according to a method of the invention include those disclosed in U.S. Pat. Publication No. 2007185432, the contents of which are incorporated by reference herein.

The following Examples further illustrate the present invention but should not be construed as in any way limiting its scope.

### Exemplification

### EXAMPLE 1

### In Vivo Iontophoretic Delivery of Salmon Calcitonin Across Microporated Skin

Purpose: To determine the effect of iontophoresis and its combination with microneedles on the in vivo delivery of salmon calcitonin (SCT) as a model peptide.
Methods: Microneedles, iontophoresis and the combination were investigated for their effect on the transdermal delivery of SCT in vivo using the hairless rat. SCT (350 μl of a 1 mg/ml solution in 50 mM citrate buffer, pH 4.0) was placed in a cartridge designed for iontophoresis. Maltose microneedles (500 micron, Texmac Inc.), stacked in three layers, were used to porate the skin prior to the application of the drug with or without iontophoresis. Since SCT (pI 10.4) was positively charged at pH 4, constant current iontophoresis (0.2 mA/cm², 1 hr) was conducted with the anode connected to the cartridge, and the cathode connected to a TransQ (IOMED, Inc.) inactive electrode. Transport of drug across the skin was assessed by collecting blood samples at regular intervals via the tail vein which were analyzed for serum SCT using ELISA.
Results: The maximum concentrations of SCT in the serum were 41.45 pg/ml, 605.21 pg/ml, and 2374.06 pg/ml under microneedles alone, 1 hr iontophoresis alone, and the combination, respectively. When compared to the delivery with microneedles alone, the increase in concentration with iontophoresis alone was 15-fold (p<0.05) and with the combination of microneedles the increase was 57-fold (p<0.05). The total amount of SCT delivered by iontophoresis and its combination with microneedles in the hairless rat was 648.67 ng/kg and 3075.96 ng/kg, respectively, as calculated by WinNonlin.
Conclusion: Iontophoresis or a disruption of the skin barrier by microneedles enabled the transdermal delivery of SCT. A combination of iontophoresis and microneedles resulted in the highest delivery flux.

### EXAMPLE 2

### In Vivo Delivery of Salmon Calcitonin Using Iontophoresis in Combination with Microporation Using Titanium Needle Arrays

Titanium needles with a width, thickness and height of 150 um, 75 um and 750 um, respectively, in arrays of 24 needles (6×4) with 0.65″ center to center spacing were used to porate the skin prior to application of SCT. SCT was measured after application of microporation alone, iontophoresis alone and microporation in combination with iontophoresis. SCT was delivered and measured as described above in Example 1.

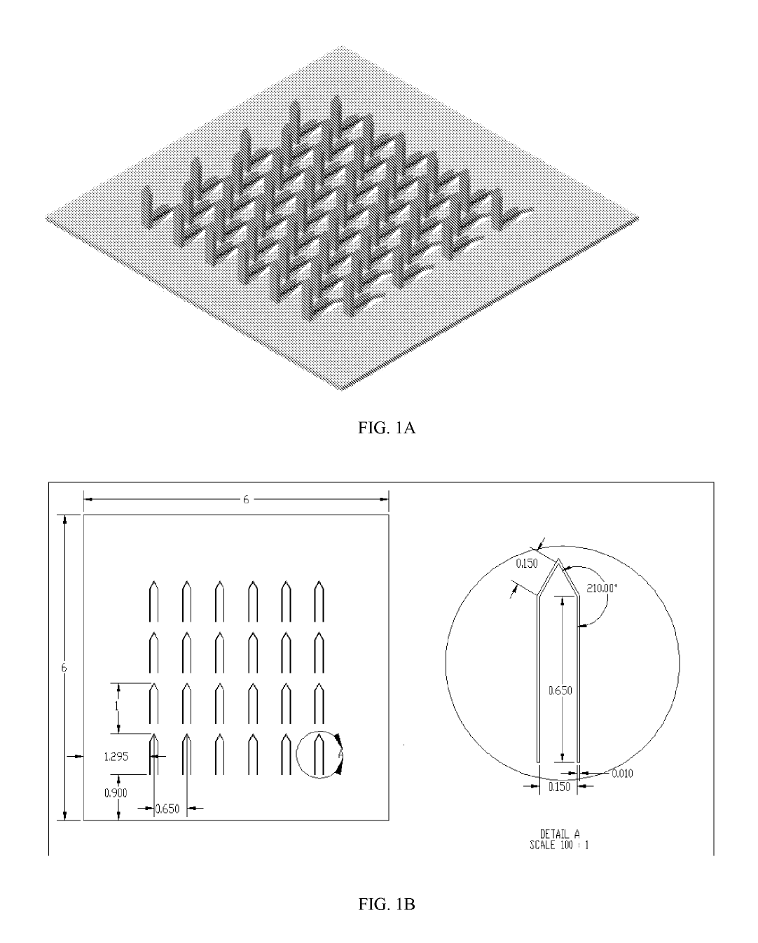

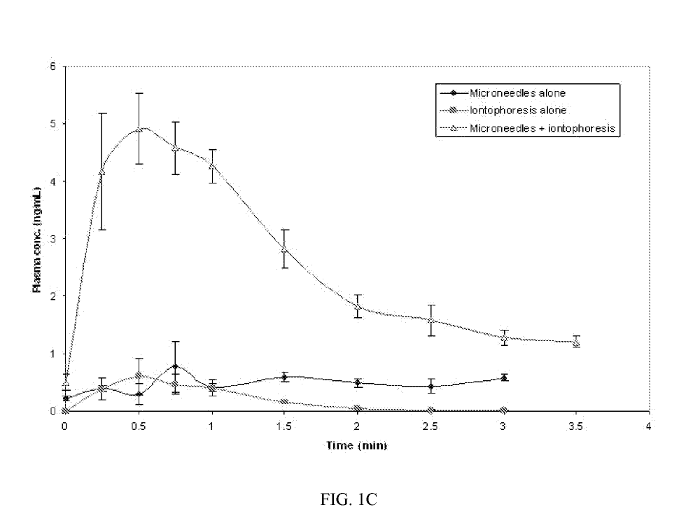

FIG. 1A is a drawing of the array bent out of the plane and FIG. 1B shows the dimensions of the needle and the array. AS shown in FIG. 1C, the plasma concentration of SCT 0.5 minutes after administration using microporation in combination with iontophresis was about 10-fold greater than the concentration of SCT after administration using either microporation or iontophoresis, alone.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of transdermally administering a peptide to a patient comprising microporating the skin of the patient with a microneedle while concurrently administering the peptide through the microneedle using iontophoresis.

2. The method of claim 1 wherein the microneedle is hollow.

3. The method of claim 1 wherein the microneedle is porous.

4. The method of claim 1 wherein the skin is microporated with more than one microneedle.

5. The method of claim 4 wherein the skin is microporated with at least about one hundred microneedles.

6. The method of claim 1 wherein the length of the microneedle is less than about 1 mm.

7. The method of claim 1 wherein the microneedle is cylindrical.

8. The method of claim 7 wherein the diameter of the microneedle is about 100 to about 200 μm.

9. The method of claim 1 wherein the microneedle is non-cylindrical.

10. The method of claim 9 wherein the cross-sectional length of the microneedle is about 100 to about 200 μm.

11. The method of claim 1 wherein said microneedle is attached to or protruding from the surface of a substrate.

12. The method of claim 11 wherein the substrate is made of a flexible material.

13. The method of claim 7 wherein the length of said one or more of microneedles is about 150 to about 900 μm.

14. The method of claim 13 wherein the length of the microneedles is about 300 to about 800 μm.

15. The method of claim 1 wherein a current density from about 0.1 mA/cm² to about 0.5 mA/cm² is applied.

16. The method of claim 15 wherein the current is applied for about 5 minutes to about 2 hours.

17. The method of claim 1 wherein the peptide is present in a composition comprising a pharmaceutically acceptable excipient.

18. The method of claim 17 wherein the composition further comprises a permeation enhancer.

19. The method of claim 1 wherein the microneedle is made of a material that dissolves upon contact with fluid within the skin.

20. The method of claim 19 wherein the material is a sugar.

21. The method of claim 1 wherein the peptide is a therapeutic protein.

22. The method of claim 1 wherein the peptide has a molecular weight of at least about 1000 Da.

23. The method of claim 1 wherein the peptide has a molecular weight of at least about 3000 Da.

24. A method of transdermally administering a peptide to a patient comprising pretreating the patient's skin with microporation using a microneedle followed by administration of said peptide using iontophoresis.

25. The method of claim 24 wherein the microneedle is a solid microneedle.

26. The method of claim 25 wherein the skin is microporated with more than one microneedle.

27. The method of claim 25 wherein the microneedle is made of a material selected from the group consisting of a plastic or a metal.

28. The method of claim 24 wherein the length of the microneedle is about 150 to about 900 um.

29. The method of claim 24 wherein a current density from about 0.1 mA/cm² to about 0.5 mA/cm² is applied.

30. The method of claim 24 wherein the peptide is a therapeutic protein.

31. The method of claim 24 wherein the peptide has a molecular weight of at least about 1000 Da.

32. The method of claim 31 wherein the peptide has a molecular weight of at least about 3000 Da.
